(19) United States
(12) Patent Application Publication
(10) Pub. No.: US 2015/0130099 A1
(43) Pub. Date: May 14, 2015

(54) FORMATION METHOD OF A MIDDLE EAR PROSTHESIS

(71) Applicant: AKADEMIA GORNICZO-HUTNICZA IM. STANISLAWA STASZICA W KRAKOWIE, Krakow (PL)

(72) Inventors: Magdalena Ziabka, Zabierzow (PL); Jan Chlopek, Krakow (PL); Anna Mertas, Gliwice (PL); Wojciech Krol, Tarnowskie Gory (PL); Elzbieta Menaszek, Krakow (PL); Agnieszka Morawska, Skala (PL)

(73) Assignee: AKADEMIA GORNICZO-HUTNICZA IM. STANISLAWA STASZICA W KRAKOWIE, Krakow (PL)

(21) Appl. No.: 14/405,340

(22) PCT Filed: May 28, 2013
(86) PCT No.: PCT/PL2013/000069

§ 371 Date: Dec. 3, 2014

(30) Foreign Application Priority Data
Jun. 6, 2012 (PL) P.399439

(51) Int. Cl. B29C 33/72; B29B 9/06; A61F 2/18

(52) U.S. Cl. CPC B29C 33/72; A61F 2/18; B29B 9/06; A61F 2240/001; A61F 2002/183; A61F 2210/0071; B29C 2033/725; B29K 2023/065
USPC 264/39

(57) ABSTRACT

The process of preparing a middle ear prosthesis of polymeric material by injection moulding technology according to the presented invention is characterized by drying the thermoplastic polymer granules, preferably high density polyethylene or polyamide or polysulphone, at a temperature in the range of 60 to 150° C. for a period of 2 to 8 hours. Subsequently granules are plasticized and then injected at a temperature between 180 and 400° C. into the mold heated to a temperature between 60 and 150° C. Then obtained moulder is cooled and in the final stage it is hermetically packed in a vacuum and sterilized. In addition, the granules of the thermoplastic polymer composite are used, obtained by addition of bactericidal additive introduced into the thermoplastic polymer granules, preferably a silver powder with a particle size from 15 to 100 nm, in an amount of 0.1-3 wt %.

The scope of present invention is a method of creating a middle ear prosthesis designed for ossicular chain reconstruction.

The middle ear transmission system is damaged in most cases by chronic inflammation. The conductive hearing loss is a consequence of such a damage. Such hearing loss might be also caused by mechanical factors affecting parts or the whole of ossicular bones chain or it is a result of a birth defect. Partially or completely destroyed or damaged chain of ossicular bones requires surgery treatment basing on tympanoplasty methods consisting of the reconstruction of the continuity of ossicular chain (malleus, incus, stapes) transplanted or repaired by using alloplastic prostheses. A type of used prosthesis depends on the type of damage, distortion or immobilization. According to the literature, specifications of inventions and gathered practical data, prosthesis used in modern transplantation differ in shape, scale and material composition. Various postoperative reactions might be observed depending on the construction material. However, the modification material as far as the chemical composition may result in different postoperative reaction and give the opportunity to change biological and mechanical parameters and functions of prostheses.

In case of a complete damage of ossicular chain the Total Ossicular Replacement Prosthesis (TORP) is used. TORP prosthesis replaces all three bones. This prosthesis is implemented between the retain stapes footplate and tympanic membrane or transplant mainly during the myryngoplasty (i.e. replacement of the tympanic membrane). The prosthesis is created of a thin shaft supported on one end in the so-called head plate linking the prosthesis to tympanic membrane and on the other end equipped with so-called cup or bell linking to the stapes capitulum or leaning on stapes superstructure if stapes capitulum is damaged. The shaft length depends on the scale of ossicles chain damage and on a patient's anatomy. A supporting part is designed as a compact pad or an openwork head plate and its diameter equals 2 mm. Functional construction of head plate has been designed in such a shape that allows for a precise positioning of the implant and simplifies the implementation process during surgery. A cup is 1 mm high and thanks to its shape allows for introducing remain healthy bone (eg. stapes capitulum) into prosthesis. TORP prostheses are known from US Patent application publications US20090164010 and US20110054607. Prostheses, and their parts, are made of pure titanium, gold, tantalum and their alloys, or from materials with shape memory such as Nitinol, ceramic or biocompatible polymers such as silicone and polytetrafluoroethylene (PTFE) and as well as the composite materials containing carbon fibers. It is also known from U.S. Pat. No. 3,909,852 patent the middle ear prosthesis in which the polytetrafluoroethylene with carbon fibers has been used as a supporting part while piston is made of biocompatible polymers such as polytetrafluoroethylene or high density polyethylene.

In case of chain reconstruction, in which at least, one bone malleus or stapes is not damaged, the PORP prostheses are used. Such prostheses replace part of chain and is located between malleus and stapes. As it is known from Polish patent publication PL156476 polymer prosthesis is used in case of improper growth or dysfunction of stapes. The hitching part (wire) is made of metal (tantalum) and allows for linking prosthesis on long process of the incus or on first ossicle bone if long process of the incus or incus are absent. In such a way the transmission system is rebuild. As an alternative, Australian patent publication AU2011202466 introduced PORP prosthesis composed of head plate and clamp (so called “bell”) attaching prosthesis to existing stapes capitulum. A middle part (counterpart of piston in other prosthesis) has been replaced with adjustable and clenching tape which locks and sticks prosthesis to the bone.

Using prosthesis during surgery operation should not require difficult implementation procedures. Moreover, it should be also possible to adjust prosthesis during the operation to a replaced part of ossicular chain. Openwork head plate construction of prosthesis allows to place it properly in the middle ear. Due to a different level of damage of ossicular chain it is often necessary to adjust prosthesis during an implementation process. It is possible to curve the bell to stapes capitulum or length adjustment; this affects not only a better adaptation to a replacing part of ossicles bones but also makes the implementation process easier and less susceptible to surgery compilations. It is possible to regulate prosthesis on two sections. In case of a metal prosthesis it is possible to adjust the length between head plate and bell. Such regulation is known from German patent publication DE102009016468. Head plate and bell are joined with an adjustable piston allowing to change its axial length. In case of polymer prosthesis, due to their chemical and physical properties, it is possible to regulate length of the whole implant by cutting lower part of prosthesis, that is bell and proper attachment to existing bone.

Modern implants not only support regeneration process of tissues and improve integration of implant with bones but also prevent recurring diseases and inflammations caused by the presence of undesirable microorganisms. Achieving such a level of implant properties is possible by enriching materials used or by adding bioactive, biostatic or bactericidal additives. According to US20080234817 and U.S. Pat. No. 5,578,086 patents publications the prostheses made of silicone and polyurethane are modified with bioactive ceramic (hydroxyapatite or bioglass). Bioactive particles are dispersed in polymer matrix or exist as a coating on a prosthesis surface. Also antibacterial coatings are used to prevent bacteria proliferation in the middle ear. They have been widely described in publication of P. Gong , H. Li, X. He et. al. “Preparation and antibacterial activity of Fe₃O₄ and Ag nanoparticles” (Nanotechnology 18 (2007), 604-11). Such coatings contain nanoparticles of copper, zinc, titanium, magnesium, gold and silver. However, the silver ones show the highest effectiveness against bacteria, fungi, viruses and other microorganisms. US2011272276 patent publication concerns the antibacterial coatings releasing silver ions to a human body in a controlled process what makes them possible to be used in any prosthesis applied in surgery. It is also popular to enrich prosthesis materials with medicaments. US20090088844 patent publication describes drug-eluting stapes prosthesis consisting of piston made of polymer and hook shaped metal wire. The piston contains drug which after implementation to the body is delivered to surrounding tissues in a controlled way.

Method of producing a middle ear prosthesis made of polymer materials by injection moulding technology according to the invention is characteristic due to the fact that granules of thermoplastic polymer are dried in temperature between 60 and 150° C. for 2 to 8 hours. In the next stage it needs to be plasticized and injected with the temperature between 180 to 400° C. to the mold heated to the temperature between 60 to 150° C. Thus obtained the moulder needs to be cooled and finally hermetically packed in a vacuum and sterilized with use of low temperature plasma with application of hydrogen peroxide vapour.

It is also beneficial to use high density polyethylene or polyimide or polysulphone as a thermoplastic polymer.

Moreover, as a thermoplastic polymer the composite granules obtained by introduction to thermoplastic polymer granules bactericidal modifying additive in amount of 0.1-3 wt % is used. Subsequently the homogenization of composite mixtures, plasticization, extrusion, cooling and finally the mechanical grinding are performed.

In certain situations an additive of silver powder with particles between 15 and 100 nm is used as an antibacterial modifier.

Thanks to thermoplastic polymers used as materials designed for reconstruction of ossicular bones, the middle ear prosthesis produced according to this invention is characteristic for its light weight and its length adjustable according to the chain replacement. The required length is obtained during surgery by cutting prosthesis. Openwork head plate of prosthesis gives the opportunity for precise implementation during the operation and easies surgeon work. Moreover, round shape of head plate minimizes the risk of damage of tympanic membrane. Addition of bactericidal additive of silver nanoparticles to the polymer matrix allows for revealing a new antibacterial function of the prosthesis against bacteria and fungi.

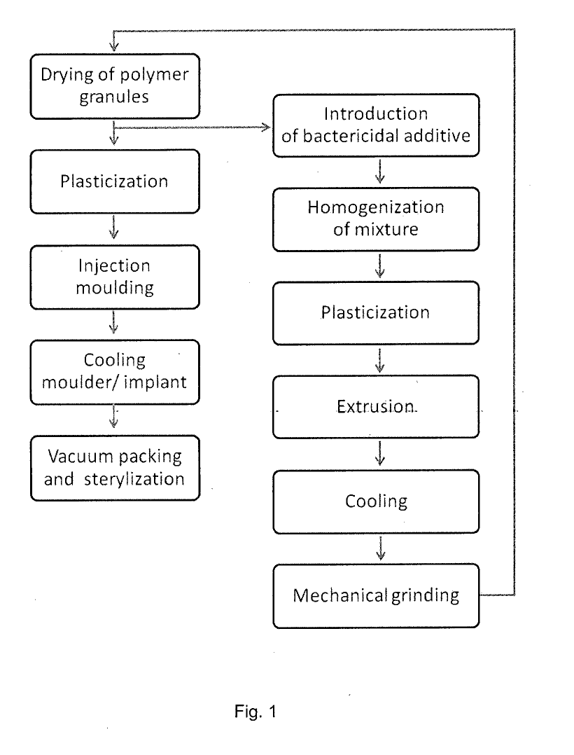

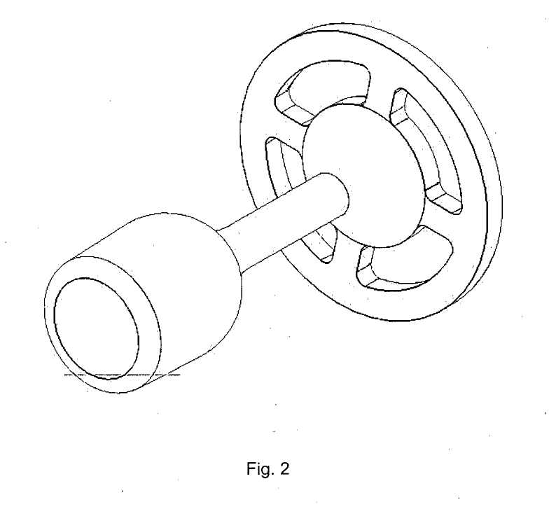

To understand a described invention fully it is important to get familiar with a below description of an exemplary technological process showed in [FIG. 1]. FIG. 2 shows a prospective view of TORP middle ear prosthesis, produced according to a described invention.

### Example 1

Granules of high density polyethylene (HDPE) with the molecular mass of 5000 g/mol and a melting point in the range of 180-220° C. are dried at 100° C. for 2 hours in the laboratory dryer. Then the granules are plasticized during two minutes in the chamber of injection moulder at 200° C., and then injected at temperature of 205° C. under the pressure of 80 kg/cm² and 60% flow into the mold, heated to 90° C. The obtained moulder is cooled to temperature ca. 60° C., removed from the mold and allowance of polymer is eliminated. In the final stage the moulder is hermetically packed in a vacuum and sterilized with the use of low temperature plasma with application of hydrogen peroxide vapour.

### Example 2

Granules of high density polyethylene (HDPE) with the molecular mass of 5000 g/mol and a melting point in the range of 180-220° C. are dried at 100° C. for 2 hours in the laboratory dryer. Then, 2 wt. % of silver nanopowder (Ag) with the mean particle size of 26 nm is added. The mixture is homogenized by mechanical shaking for 20 minutes and then plasticized and continuously extruded in the form of stream, which is cooled to room temperature and mechanically ground using a grinder equipped with cutting blades. Composite granules are dried at 100° C. for 2 hours in the laboratory dryer, for 3 minutes plasticized in the chamber of injection moulder at the temperatures of 200° C., and then injected at a temperature of 210° C. under a pressure of 80 kg/cm² and the flow rate of 60% into a mold heated to a temperature of 100° C. The obtained moulder is cooled to temperature ca. 60° C., removed from the mold and allowance of polymer is eliminated. In the final stage the moulder is hermetically packed in a vacuum and sterilized with the use of low temperature plasma with application of hydrogen peroxide vapour, as in example 1. A simple structure of high density polyethylene and well distributed silver in the matrix significantly influence the silver ions released into the environment, thereby increasing the bactericidal effectiveness of composites. This is confirmed by the tests carried out against the standard strains of Gram-positive bacteria (Staphylococcus aureus ATCC 25923) and Gram-negative bacteria (Escherichia coli ATCC 25922). The results are shown in the following table.

| | Staphylococcus aureus | | Escherichia coli | |
|---|---|---|---|---|
| | ATCC 25923 | | ATCC 25922 | |
| | (initial density | | (initial density | |
| | 1.5 × 10⁵ CFU/ml) | | 1.5 × 10⁵ CFU/ml) | |
| | CFU/ml after | | CFU/ml after | |
| | 17 hours of | Anitbacterial | 17 hours of | Anitbacterial |
| | incubation | Efficacy ABE | incubation | Efficacy ABE |
| Material | with material | (%) | with material | (%) |
| HDPE | 5.6 × 10⁷ | 6.67 | 1.35 × 10⁸ | 10.0 |
| HDPE/2 | 1.0 × 10⁴ | 99.9 | 1.4 × 10³ | 99.9 |
| wt. % Ag | | | | |
| BLANK | 6.0 × 10⁷ | — | 1.5 × 10⁸ | — |

*CFU—colony forming units (concerns bacterial suspension in tryptonic water with the colony-forming bacteria).

What is claimed is:

1-4. (canceled)

5. A method of producing a middle ear prosthesis, wherein the middle ear prosthesis comprises a polymer material, said method comprising the steps of
a) performing a drying process of the polymer material to form a thermoplastic polymer granulate,
b) performing a plasticizing process on the thermoplastic polymer granulate and injecting the thermoplastic polymer granulate into a preheated mold to form the prosthesis,
c) subjecting the mold to a hermetic vacuum coating with film, and
d) subjecting the mold to a low temperature plasma sterilization with hydrogen peroxide vapor,
wherein the thermoplastic polymer granulate is modified via addition of a bactericidal additive in amount between about 0.1 to about 3 wt. %.

6. The method of claim 5, wherein the polymer material is selected from the group consisting of a high-density polyethylene, a polyamide, and a polysulfone.

7. The method of claim 5, wherein the bactericidal additive is silver powder.

8. The method of claim 7, wherein the silver powder has a particle size of about 15 nm to about 100 nm.

9. The method of claim 5, wherein the thermoplastic polymer granulate is subjected to homogenization.

10. The method of claim 5, wherein the thermoplastic polymer granulate is subjected to plastification.

11. The method of claim 5, wherein the thermoplastic polymer granulate is subjected to continuous extrusion

12. The method of claim 5, wherein the thermoplastic polymer granulate is subjected to chilling.

13. The method of claim 5, wherein the thermoplastic polymer granulate is subjected to mechanical grinding.
